(19) United States
(12) Patent Application Publication
(10) Pub. No.: US 2020/0164175 A1
(43) Pub. Date: May 28, 2020

(54) SYSTEM FOR TREATING DEPRESSION USING COMPLEX BIOMETRIC DATA

(71) Applicant: Blaubit Co., Ltd., Seongnam-si (KR)

(72) Inventor: Euisun KIM, Seoul (KR)

(73) Assignee: Blaubit Co., Ltd., Seongnam-si (KR)

(21) Appl. No.: 16/227,414

(22) Filed: Dec. 20, 2018

(30) Foreign Application Priority Data
Nov. 26, 2018 (KR) 10-2018-0146908

(51) Int. Cl. A61M 21/00; G06F 3/01; A61B 5/0205; A61B 5/00; A61G 15/00; A61G 15/02; A61B 5/16

(52) U.S. Cl. CPC A61M 21/00; G06F 3/015; A61B 5/02055; A61B 5/6803; A61B 5/024; A61G 15/02; A61B 5/165; A61M 2021/0027; A61M 2021/005; A61G 15/007

(57) ABSTRACT

Disclosed is a system for treating depression using complex biometric data. The present disclosure controls the operation of a 4D chair according to complex biometric data of a patient, which is measured in association with the reproduction of VR contents for depression treatment, analyzes the measured complex biometric data, and displays the depression improvement state of the patient.

### CROSS-REFERENCE TO RELATED APPLICATION(S)

This application claims the benefit under 35 USC 119(a) of Korean Patent Application No. 10-2018-0146908 filed on Nov. 26, 2018, in the Korean Intellectual

Property Office, the entire disclosure of which is incorporated herein by reference for all purposes.

### BACKGROUND OF THE INVENTION

### 1. Field of the Invention

The present disclosure relates to a system for treating depression using complex biometric data and, more particularly, to a system for treating depression using complex biometric data, which controls the operation of a 4D chair according to complex biometric data on a patient, which is measured in association with reproduction of VR contents for depression treatment, analyzes the measured complex biometric data, and displays the depression improvement state of the patient.

### 2. Description of the Prior Art

In general, depression (or depressive disorder) refers to illness that causes a variety of cognitive and psychosomatic symptoms, such as low motivation and melancholy as primary symptoms, thereby impeding daily physical functions.

In addition, the number of women who suffer from depression is double the number of men who suffer from the same. Although women generally have a higher serotonin level than men, women are more likely to suffer from depression because they are very sensitive to a small change in the serotonin level.

In addition, the probability of suffering from depression during one's lifetime is about 15%, in particular, about 25% for women, and is a serious disease that causes changes in emotion, thought, physical condition, and behavior.

Depression is different from temporary gloominess, is not a sign of personal weakness, and cannot be overcome through individual will. Most patients endure depression without seeking professional help, but if they receive proper care from professionals, it is possible to expect a significant improvement and to return to the previous normal life.

Korean Patent No. 10-1626834 (entitled “Depression monitoring system and method based on user data”) discloses a system for monitoring depression on the basis of user data, which includes a user data collection module for collecting user activity data or biometric data in real time and a user data analysis module for analyzing data collected by the user data collection module in real time and quantifying the degree of depression, thereby automatically monitoring depression in user's daily activities, and further discloses a method thereof.

However, in the depression monitoring system and method according to the prior art described above, the configuration of quantifying the degree of depression cannot provide proper treatment for depression depending on the patient's depression status, and cannot provide the improvement state of depression.

### SUMMARY OF THE INVENTION

In order to solve the problems described above, an aspect of the present disclosure provides a system for treating depression using complex biometric data, which controls the operation of a 4D chair according to complex biometric data of a patient, which is measured in association with reproduction of VR contents for depression treatment, analyzes the measured complex biometric data, and displays the depression improvement state of the patient.

In view of the above aspect, the present disclosure includes: a terminal configured to, when a patient wears the terminal while sitting on a chair, output pre-stored virtual reality (VR) content data for depression treatment, detect biometric data of the patient who responds to the output VR content data, output an operation control signal so as to change the position and shape of the chair on which the patient is sitting according to the detected biometric data, normalize the detected biometric data of the patient, thereby calculating predetermined individual index data, analyze the depression treatment state of the patient using the calculated index data, and display the analysis result; and a chair configured to change the position and shape thereof according to the operation control signal transmitted from the terminal, while the patient is sitting on the chair, such that the sitting position of the patient is changed.

In addition, the terminal according to the present disclosure is a head-mounted display (HMD).

In addition, the terminal according to the present disclosure includes: a biometric data sensor configured to detect at least one of pulse waves, brain waves, body temperature, an electromyogram (EMG), and sweat of the patient; a content manager configured to output, to the patient, the virtual reality (VR) content data for depression treatment containing video and sound data; a biometric data analyzer configured to analyze the biometric data detected by the biometric data sensor, thereby calculating a biometric value of the patient responding to the output VR content data, output the operation control signal according to the calculated biometric value so as to change the position and shape of the chair on which the patient is sitting, normalize the detected biometric data of the patient using a stored program, thereby calculating the predetermined individual index data, analyze the depression treatment state of the patient by comparing the calculated index data with reference data, and display the analysis result; a display configured to display the VR content data for depression treatment of the content manager and the depression treatment state data of the biometric data analyzer; and an audio output part configured to output the VR content data for depression treatment by the content manager.

In addition, the biometric data analyzer is configured to allow the normalized biometric data of the patient to be displayed as at least one of a stress index, a concentration index, and a psychological stability index.

Furthermore, the chair is a 4-dimensional (4D) chair that includes: a chair controller configured to output a driving signal according to the operation control signal for a change in the position and shape of the chair, which is received from the terminal; and a chair driving part configured to provide driving force according to the driving signal such that the position and shape of the chair are changed.

The present disclosure can relieve the patient's tension and can improve the psychological stability and concentration ability of the patient by obtaining complex biometric data, such as brain waves, pulse waves, body temperature, etc. of a patient in association with reproduction of VR contents for depression treatment and by controlling the operation of a 4D chair in real time according to the obtained biometric data.

In addition, the present disclosure calculates a stress index, a concentration index, a psychological stability index, etc. of the patient from the complex biometric data obtained during the treatment and displays the depression improvement state of the patient, thereby improving the patient's satisfaction for treatment.

### BRIEF DESCRIPTION OF THE DRAWINGS

The above and other aspects, features, and advantages of the present disclosure will be more apparent from the following detailed description taken in conjunction with the accompanying drawings, in which:

FIG. 1 is an exemplary view illustrating a system for treating depression using complex biometric data according to the present disclosure;

FIG. 2 is an exemplary view illustrating the use state of a system for treating depression using complex biometric data according to the present disclosure;

FIG. 3 is a block diagram illustrating the configuration of a system for treating depression using complex biometric data according to the present disclosure; and

FIG. 4 is a block diagram illustrating the configuration of a terminal in a system for treating depression using complex biometric data according to the present disclosure.

### DETAILED DESCRIPTION OF THE EXEMPLARY EMBODIMENTS

Hereinafter, a preferred embodiment of a system for treating depression using complex biometric data according to the present disclosure will be described in detail with reference to the accompanying drawings.

In the present specification, the expression “a part includes an element” does not mean that the part excludes other elements, but means that the part may further include other elements.

In addition, the term “part”, “-er (or)”, “module”, or the like means a unit for processing at least one function or operation, and may be implemented as hardware, software, or a combination thereof.

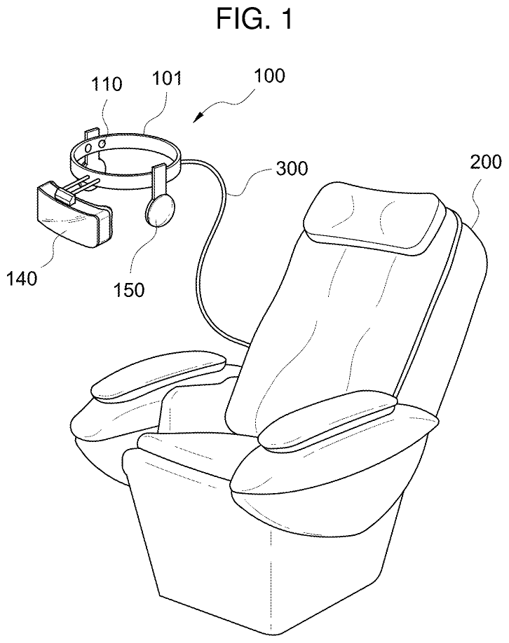

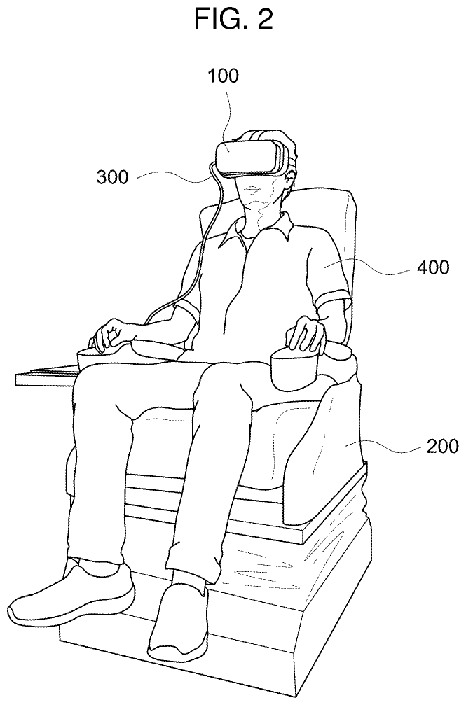

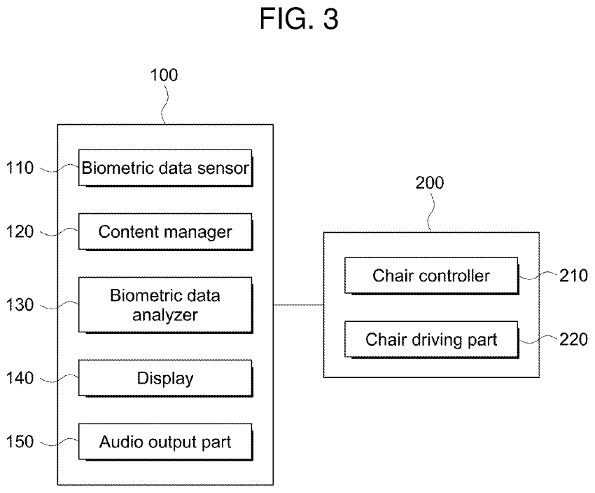

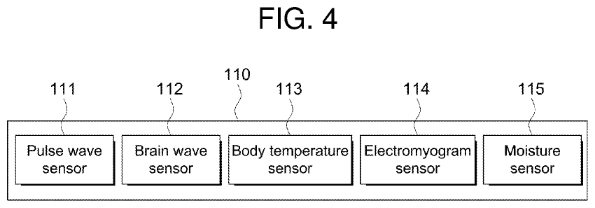

FIG. 1 is an exemplary view illustrating a system for treating depression using complex biometric data according to the present disclosure, and FIG. 2 is an exemplary view illustrating the use state of a system for treating depression using complex biometric data according to the present disclosure. FIG. 3 is a block diagram illustrating the configuration of a system for treating depression using complex biometric data according to the present disclosure, and FIG. 4 is a block diagram illustrating the configuration of a terminal in a system for treating depression using complex biometric data according to the present disclosure.

As shown in FIGS. 1 to 4, a system for treating depression using complex biometric data, according to the present disclosure, is configured to include a terminal 100 that is worn on a patient 400 so as to output virtual reality (VR) content data for depression treatment and a chair 200 that is connected to the terminal 100 so as to change the sitting posture of the patient 400 while the VR content data is output.

The terminal 100 is configured to: output pre-stored VR content data for depression treatment when it is worn on the patient 400 sitting on the chair 200; detect complex biometric data of the patient 400 who responds to the output VR content data; output an operation control signal depending on the detected complex biometric data so as to change the position and shape of the chair 200 on which the patient 400 is sitting; calculate predetermined individual index data by normalizing the detected biometric data of the patient 400; analyze the depression treatment state of the patient 400 using the calculated index data; and display the analysis result, and is configured to include a biometric data sensor 110, a content manager 120, a biometric data analyzer 130, a display 140, and an audio output part 150.

In addition, the terminal 100 is preferably configured as a head-mounted display (HMD) device in which the biometric data sensor 110, the content manager 120, the biometric data analyzer 130, the display 140, and the audio output part 150 are integrated by means of a head strap 101.

That is, the head strap 101, which is worn on the head of the patient 400, has the biometric data sensor 110, the display 140, and the audio output part 150 provided thereon, and the display 140 has the content manager 120 and the biometric data analyzer 130 provided in an on-board form.

The biometric data sensor 110 is intended to detect biometric data of the patient in relation to pulse waves, brain waves, body temperature, electromyogram (EMG), and sweat of the patient 400, and is configured to include a pulse wave sensor 111 for measuring a wave signal output in the form of a wave by blood in the heart (i.e., pulse wave signal), a brain wave sensor 112 for measuring brain wave signals, such as delta, theta, alpha, SMR, low beta, high beta, gamma, or the like, a body temperature sensor 113 for measuring the body temperature of the patient 400, an electromyogram sensor 114 for measuring an activity current of skeletal muscle of the patient 400, and a moisture sensor 115 for measuring secretions such as sweat.

In addition, the biometric data sensor 110, which is a photoplethysmograph (PPG) measurement device, measures biometric data of the patient 400 by a non-invasive method.

The content manager 120 is configured to output, to the patient 400, virtual reality (VR) content data for depression treatment including video and sound data on the display 140, wherein the displayed VR content data may vary depending on the patient to be treated and the degree of depression thereof, and wherein necessary VR content data may be transmitted from external terminals (not shown), such as PCs or servers, through a network for connection, to then be stored and output.

The VR content data may include a variety of data, such as documentary videos and sounds including lectures, performances, nature scenes, and the like, which can induce psychological stability of the patient and can relieve a patient's tension.

The biometric data analyzer 130 analyzes the biometric data detected by the biometric data sensor 110, performs control so as to change the position and shape of the chair 200, on which the patient 400 is sitting, depending on the analysis result, and analyzes the depression treatment state of the patient 400 on the basis of the detected biometric data, thereby outputting the same.

That is, when the VR content data is output by the content manager 120, the biometric data analyzer 130 obtains, from the biometric data sensor 110, biometric data of the patient 400 who responds to the output VR content data, and calculates a biometric value by quantifying the degree of real-time response of the patient 400 to the VR contents.

In addition, the biometric data analyzer 130 outputs an operation control signal for changing the position and shape of the chair 200 depending on the calculated biometric value in order for the patient 400 to have a more comfortable and psychologically stable posture.

The biometric data analyzer 130 enables various changes in the position and shape of the chair 200 such as, for example, changing the backrest position of the chair 200 such that the upper body of the patient 400 is reclined backwards at a specific angle while sitting on the chair 200 or adjusting the overall slope of the chair 200 to a flat state such that the patient 400 switches from the seated state to the lying state.

That is, along with the treatment using the VR contents, the posture of the patient is changed to stay in an optimal state using the chair 200 in order to promote the psychological stability of the patient 400, thereby increasing the treatment effect of the patient 400.

In addition, the biometric data analyzer 130 may be configured to output operation control signals in synchronization with the time frame of the displayed VR content data such that the patient 400 can feel the wind, the smells, the heat, or the like through the chair 200, thereby further improving the treatment effect of the patient 400 using the somatosensory function of the patient 400.

In addition, the biometric data analyzer 130 normalizes the detected biometric data of the patient 400 by means of a pre-stored analysis program, thereby calculating predetermined individual index data.

That is, the biometric data analyzer 130, for example, calculates the pulse wave difference from the measured pulse wave signal, performs normalization from the calculated pulse wave difference through frequency analysis, calculates real-time biometric data of the patient 400 as a stress index, a concentration index, a psychological stability index, and the like by comparison and analysis with normalized signals of other measured signals, such as a brain wave signal, a body temperature signal, an electromyogram signal, a sweating signal, or the like, and displays the calculated stress index, concentration index, and psychological stability index.

In addition, the biometric data analyzer 130 compares the calculated stress index, concentration index, and psychological stability index with pre-stored reference data or previous treatment data of the patient 400, thereby analyzing the depression treatment state of the patient 400, and the analysis result is stored in a memory (not shown) to then be displayed on the display 140.

The display 140 is configured to display video data contained in the VR content data for depression treatment, which is output from the content manager 120, and the depression treatment state and the analysis result of the biometric data analyzer 130 to the patient 400, and includes displaying means such as an LCD, an LED, or the like.

The audio output part 150 is configured to output sound data contained in the VR content data for depression treatment, which is output from the content manager 120, to the patient 400, and includes a speaker or the like.

The chair 200 is configured to allow the patient 400 to sit thereon and is connected to the terminal 100 through a cable 300. The chair 200 includes a seat, a backrest, an armrest, a leg rest, and a headrest for supporting the hips, back, arms, legs, and head of the patient 400, respectively. In addition, the chair 200 is configured to include a chair controller 210 and a chair driving part 220, which change the position and shape of the chair so as to change the sitting posture of the patient 400 according to an operation control signal transmitted from the terminal 100 while the patient 400 is sitting on the chair 200.

Although the present embodiment describes that the terminal 100 and the chair 200 are connected by the cable 300, the present disclosure is not limited thereto, and they may be connected by means of wireless data communication such as Bluetooth.

When an operation control signal for changing the position and shape of the chair 200 is received from the terminal 100, the chair controller 210 outputs a driving signal corresponding to the received operation control signal to the chair driving part 220 such that the configurations (e.g., the positions of the seat, the backrest, the armrest, the leg rest, and the headrest) of the chair 200 are changed, or such that the overall slope of the chair 200 is adjusted or the chair 200 becomes flat like a bed as to the shape thereof in order for the patient 400 to have a lying posture.

The chair driving part 220 is configured to provide driving force according to the driving signal output from the chair controller 210 such that the positions of the seat, the backrest, the armrest, the leg rest, and the headrest, the slope of the chair 200, or the shape of the chair 200 are changed, and includes a plurality of driving means such as an actuator, a motor, and the like.

In addition, the chair driving part 220 may further include a blower for providing wind, smell, or the like and a heating wire for providing heat, thereby configuring a 4-dimensional (4D) chair capable of proving the patient 400 with a treatment function using tactile stimulation.

Therefore, it is possible to relieve the patient's tension and to improve the psychological stability and concentration ability of the patient by obtaining complex biometric data of the patient, such as brain waves, pulse waves, and body temperature, in association with reproduction of VR contents for depression treatment and by controlling the operation of the chair in real time according to the obtained biometric data.

In addition, it is possible to enhance the depression improvement data of the patient and the patient's satisfaction with treatment by calculating a stress index, a concentration index, a psychological stability index, etc. of the patient from the complex biometric data, such as brain waves, pulse waves, body temperature, etc. of the patient, which are obtained during the treatment, and by displaying the depression treatment state of the patient.

While the present disclosure has been described with reference to preferable embodiments, it is to be understood that the present disclosure may be modified and changed in various forms by those skilled in the art without departing from the scope and spirit of the present disclosure as defined in the appended claims.

In addition, the reference numerals shown in the claims are merely intended for clarity and convenience of explanation, and the present disclosure is not limited thereto. The thicknesses of the lines or the sizes of the elements illustrated in the drawings of the embodiments may be exaggerated for clarity and convenience of explanation. The terms described above have been defined in consideration of functions thereof in the present disclosure, but may be varied according to the intention or practices of a user or operator, so that the interpretation of the terms must be made on the basis of the entire content of this specification.

What is claimed is:

1. A system for treating depression using complex biometric data, comprising:
a terminal (100) configured to, when a patient (400) wears the terminal (100) while sitting on a chair (200), output pre-stored virtual reality (VR) content data for depression treatment, detect biometric data of the patient (400) who responds to the output VR content data, output an operation control signal so as to change the position and shape of the chair (200) on which the patient (400) is sitting according to the detected biometric data, normalize the detected biometric data of the patient (400), thereby calculating predetermined individual index data, analyze the depression treatment state of the patient (400) using the calculated index data, and display the analysis result; and
a chair (200) configured to change the position and shape thereof according to the operation control signal transmitted from the terminal (100) while the patient (400) is sitting on the chair such that the sitting position of the patient (400) is changed.

2. The system of claim 1, wherein the terminal (100) is a head-mounted display (HMD).

3. The system of claim 2, wherein the terminal (100) comprises:
a biometric data sensor (110) configured to detect at least one of pulse waves, brain waves, body temperature, electromyogram (EMG), and sweat of the patient (400);
a content manager (120) configured to output, to the patient (400), the virtual reality (VR) content data for depression treatment containing video and sound data;
a biometric data analyzer (130) configured to analyze the biometric data detected by the biometric data sensor (110), thereby calculating a biometric value of the patient (400) responding to the output VR content data, output the operation control signal according to the calculated biometric value so as to change the position and shape of the chair (200) on which the patient (400) is sitting, normalize the detected biometric data of the patient (400) using a stored analysis program, thereby calculating the predetermined individual index data, analyze the depression treatment state of the patient (400) by comparing the calculated index data with reference data, and display the analysis result;
a display (140) configured to display the VR content data for depression treatment of the content manager (120) and the depression treatment state data by the biometric data analyzer (130); and
an audio output part (150) configured to output the VR content data for depression treatment by the content manager (120).

4. The system of claim 3, wherein the biometric data analyzer (130) is configured to allow the normalized biometric data of the patient (400) to be displayed as at least one of a stress index, a concentration index, and a psychological stability index.

5. The system of claim 1, wherein the chair (200) is a 4-dimensional (4D) chair comprising:
a chair controller (210) configured to output a driving signal according to the operation control signal for a change in the position and shape of the chair (200), which is received from the terminal (100); and
a chair driving part (220) configured to provide driving force according to the driving signal such that the position and shape of the chair (200) are changed.
